(19) United States
(12) Patent Application Publication
(10) Pub. No.: US 2020/0110257 A1
(43) Pub. Date: Apr. 9, 2020

(54) IMAGING UNIT AND ENDOSCOPE

(71) Applicant: OLYMPUS CORPORATION, Tokyo (JP)

(72) Inventors: Hiroyuki MOTOHARA, Tokyo (JP); Shinya ISHIKAWA, Tokyo (JP); Toshiyuki SHIMIZU, Tokyo (JP)

(73) Assignee: OLYMPUS CORPORATION, Tokyo (JP)

(21) Appl. No.: 16/709,136

(22) Filed: Dec. 10, 2019

Related U.S. Application Data
(63) Continuation of application No. PCT/JP2018/021190, filed on Jun. 1, 2018.

(30) Foreign Application Priority Data
Jun. 15, 2017 (JP) 2017-118073

(51) Int. Cl. G02B 23/24; H04N 5/225; H01L 27/146; G02B 5/04; A61B 1/00; A61B 1/05

(52) U.S. Cl. CPC G02B 23/2446; H04N 5/2254; H01L 27/14629; A61B 1/05; A61B 1/00096; A61B 1/00013; G02B 23/2423; G02B 5/04

(57) ABSTRACT

An imaging unit includes: an optical system including a plurality of lenses; a prism configured to reflect light condensed by the optical system; a semiconductor package including an image sensor configured to generate an electrical signal by receiving light incident from the prism and performing photoelectric conversion on the received light, and including a connection electrode on a back surface of the semiconductor package; and a multi-layer substrate including a connection terminal on a top surface of the multi-layer substrate, the connection electrode being connected to the connection electrode via a conductive member. A concave portion in which an electronic component is mounted is formed in a region on a back surface of the multi-layer substrate, the region corresponding to a region where the connection terminal is formed.

### CROSS-REFERENCE TO RELATED APPLICATION

This application is a continuation of PCT International Application No. PCT/JP2018/021190 filed on Jun. 1, 2018, which designates the United States, incorporated herein by reference, and which claims the benefit of priority from Japanese Patent Application No. 2017-118073, filed on Jun. 15, 2017, incorporated herein by reference.

### BACKGROUND

### 1. Technical Field

The present disclosure relates to an imaging unit that is arranged in a distal end of an insertion portion of an endoscope to be introduced into a subject and captures an image of an inside of the subject, and relates to an endoscope.

### 2. Related Art

In the medical field and the industrial field, endoscopes have been widely used for various examinations. Among the endoscopes, a medical endoscope has been widely used because, by introducing an elongated flexible insertion portion that is provided with an imaging apparatus at a distal end thereof into a subject, such as a patient, the medical endoscope is able to acquire an in-vivo image inside the subject without making an incision in the subject and further able to perform curative treatment by causing a treatment tool to protrude from the distal end of the insertion portion.

In the insertion portion of the endoscope as described above, a light guide, a treatment tool channel, and the like are arranged in addition to the imaging apparatus inside a cladding tube (for example, see Japanese Patent Laid-open Publication No. H9-276215).

### SUMMARY

In some embodiments, an imaging unit includes: an optical system including a plurality of lenses; a prism configured to reflect light condensed by the optical system; a semiconductor package including an image sensor configured to generate an electrical signal by receiving light incident from the prism and performing photoelectric conversion on the received light, and including a connection electrode on a back surface of the semiconductor package; and a multi-layer substrate including a connection terminal on a top surface of the multi-layer substrate, the connection electrode being connected to the connection electrode via a conductive member. A concave portion in which an electronic component is mounted is formed in a region on a back surface of the multi-layer substrate, the region corresponding to a region where the connection terminal is formed, a stepped portion is formed from a distal end side to a proximal end side on a back surface side of the multi-layer substrate so as to make a width of a first side that is located on the top surface of the multi-layer substrate and that is perpendicular to an optical axis of the optical system to become longer than a width of a second side that is located on the back surface of the multi-layer substrate and that faces the first side, and a height of the concave portion and a height of the stepped portion are equal to each other.

In some embodiments, an endoscope includes: the imaging unit; and an insertion portion including a distal end portion in which a tubular main body made of a rigid material is covered by a cladding tube, the insertion portion being configured to be inserted into a subject. The imaging unit is located in an internal space of the cladding tube of the insertion portion.

In some embodiments, an endoscope includes: the imaging unit; a frame member that has an inner wall and an outer wall formed in shapes conforming to an outer shape of the imaging unit, the frame member being configured to hold the imaging unit; and an insertion portion including a distal end portion in which a tubular main body made of a rigid material is covered by a cladding tube, the insertion portion being configured to be inserted into a subject. The imaging unit held by the frame is located in an internal space of the cladding tube of the insertion portion.

In some embodiments, an imaging unit includes: an optical system including a plurality of lenses; a prism configured to reflect light condensed by the optical system; a semiconductor package including an image sensor configured to generate an electrical signal by receiving light incident from the prism and performing photoelectric conversion on the received light, and including a connection electrode on a back surface of the semiconductor package; a multi-layer substrate including a connection terminal on a top surface of the multi-layer substrate, the connection electrode being connected to the connection electrode via a conductive member. A notch is formed on a proximal end side of the multi-layer substrate so as to make a width of a first side surface that is located on a distal end side of the multi-layer substrate and that is perpendicular to an optical axis of the optical system to become longer than a width of a second side surface on the proximal end side of the multi-layer substrate.

In some embodiments, an imaging unit includes: an optical system including a plurality of lenses; a prism configured to reflect light condensed by the optical system; a semiconductor package including an image sensor configured to generate an electrical signal by receiving light incident from the prism and performing photoelectric conversion on the received light, and including a connection electrode on a back surface of the semiconductor package; and a multi-layer substrate including a connection terminal on a top surface of the semiconductor package, the connection electrode being connected to the connection electrode via a conductive member. A notch is formed on a proximal end side on a back surface side of the multi-layer substrate so as to make a length of a first side that is located on the top surface side of the multi-layer substrate and that is parallel to an optical axis of the optical system to become longer than a length of a second side that is located on the back surface side of the multi-layer substrate and that faces the first side.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating an overall configuration of an endoscope system according to a first embodiment of the present disclosure;

FIG. 2 is a perspective view of an imaging unit that is arranged in a distal end portion of an endoscope illustrated in FIG. 1;

FIG. 3 is a perspective view of the imaging unit viewed from a direction different from FIG. 2;

FIG. 4 is a cross-sectional view of the imaging unit illustrated in FIG. 2 in a case where the imaging unit is housed in a frame member;

FIG. 5 is a diagram illustrating arrangement of built-in components on a distal end side of a bending portion of the endoscope illustrated in FIG. 1;

FIG. 6 is a front elevation view of a distal end of an endoscope according to a first modification of the first embodiment of the present disclosure;

FIG. 7 is a front view of an imaging unit according to a second modification of the first embodiment of the present disclosure in a case where the imaging unit is housed in a frame member;

FIG. 8 is a cross-sectional view of a distal end side of a bending portion of an endoscope according to the second modification of the first embodiment of the present disclosure;

FIG. 9 is a front view of an imaging unit according to a third modification of the first embodiment of the present disclosure in a case where the imaging unit is housed in a frame member;

FIG. 10 is a perspective view of an imaging unit according to a second embodiment of the present disclosure;

FIG. 11 is a perspective view of the imaging unit viewed from a direction different from FIG. 10;

FIG. 12 is a diagram for explaining a conventional imaging unit (mounting misalignment has not occurred);

FIG. 13 is a diagram for explaining a conventional imaging unit (mounting misalignment has occurred);

FIG. 14 is a diagram for explaining the imaging unit according to the second embodiment of the present disclosure (mounting misalignment has occurred);

FIG. 15 is a partial top view of a multi-layer substrate according to a first modification of the second embodiment of the present disclosure;

FIG. 16 is a partial top view of a multi-layer substrate according to a second modification of the second embodiment of the present disclosure; and

FIG. 17 is a side view of an imaging unit according to a third embodiment of the present disclosure.

### DETAILED DESCRIPTION

Hereinafter, as modes for carrying out the present disclosure (hereinafter, referred to as “embodiments”), an endoscope system including an imaging unit will be described. The present disclosure is not limited by the embodiments below. Further, in description of the drawings, the same components are denoted by the same reference symbols. Furthermore, it is necessary to note that the drawings are schematic, and a relation between a thickness and a width of each component, ratios among the components, and the like are different from actual ones. Moreover, the drawings may include portions that have different dimensions or ratios.

### First Embodiment

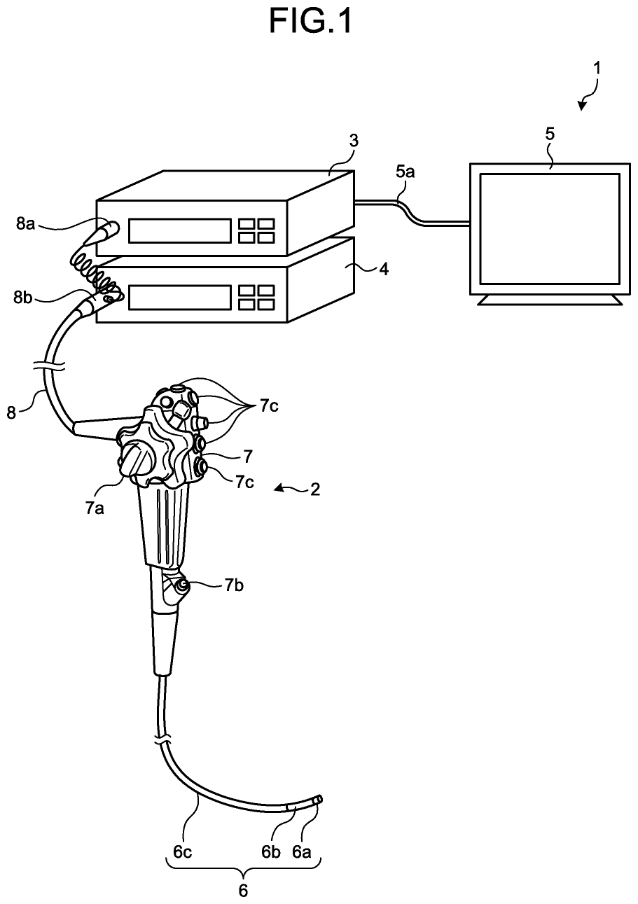

FIG. 1 is a diagram schematically illustrating an overall configuration of an endoscope system 1 according to a first embodiment of the present disclosure. As illustrated in FIG. 1, the endoscope system 1 according to the first embodiment includes an endoscope 2 that is introduced into a subject, that captures an image of an inside of the subject, and that generates an image signal inside the subject, an information processing apparatus 3 that performs predetermined image processing on the image signal obtained by imaging by the endoscope 2 and controls each of components of the endoscope system 1, a light source device 4 that generates illumination light of the endoscope 2, and a display device 5 that displays an image of the image signal subjected to the image processing by the information processing apparatus 3.

The endoscope 2 includes an insertion portion 6 that is to be inserted into the subject, an operating unit 7 that is arranged on a proximal end portion side of the insertion portion 6 and that is grasped by an operator, and a flexible universal cord 8 that is extended from the operating unit 7.

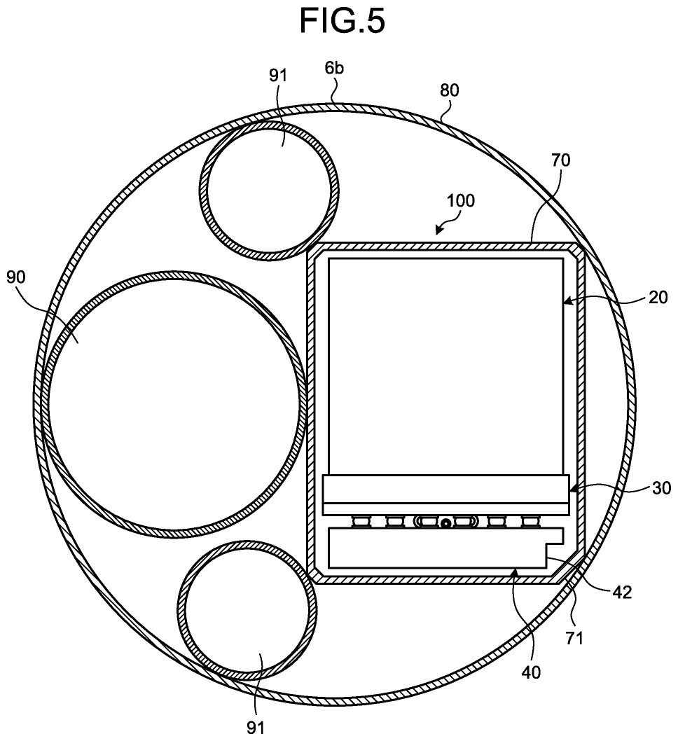

The insertion portion 6 is realized by an illumination fiber (light guide cable), an electrical cable, an optical fiber, and the like. The insertion portion 6 includes a distal end portion 6a in which an imaging unit (to be described later) is incorporated, a bending portion 6b that is constituted by a plurality of bending pieces and that is freely bendable, and a flexible tube portion 6c that is arranged on a proximal end portion side of the bending portion 6b and that has flexibility. In the distal end portion 6a, illumination channels 91 that are communicated with illumination fibers for illuminating the inside of the subject via an illumination lens, and a treatment tool channel 90 in which a treatment tool is inserted are arranged (see FIG. 5).

The operating unit 7 includes a bending knob 7a that causes the bending portion 6b to bend in the vertical direction and in the horizontal direction, a treatment tool insertion portion 7b through which a treatment tool, such as a biopsy forceps or a laser scalpel, is inserted into a body cavity of the subject, and a plurality of switch portions 7c that perform operation on peripheral devices, such as the information processing apparatus 3, the light source device 4, an air supply device, a water supply device, and a gas supply device. The treatment tool inserted through the treatment tool insertion portion 7b gets out of an opening portion at a distal end of the insertion portion 6 via the internally-arranged treatment tool channel 90 (see FIG. 5).

The universal cord 8 is constituted by an illumination fiber, a cable, or the like. The universal cord 8 is branched at a proximal end such that an end portion of one branch serves as a connector 8a and a proximal end of the other branch serves as a connector 8b. The connector 8a is freely attachable to and detachable from a connector of the information processing apparatus 3. The connector 8b is freely attachable to and detachable from the light source device 4. The universal cord 8 propagates illumination light emitted by the light source device 4 to the distal end portion 6a via the connector 8b and the illumination fiber. Further, the universal cord 8 transmits an image signal that is obtained by imaging by the imaging unit (to be described later) to the information processing apparatus 3 via a cable and the connector 8a.

The information processing apparatus 3 performs predetermined image processing on the image signal output from the connector 8a and controls the entire endoscope system 1.

The light source device 4 is constituted by a light source that emits light, a condenser lens, and the like. The light source device 4 emits light from the light source and supplies the light as illumination light for illuminating the inside of the subject as an imaging object to the endoscope 2 that is connected via the connector 8b and the illumination fiber in the universal cord 8, under the control of the information processing apparatus 3.

The display device 5 is constituted by a display or the like made with liquid crystal or organic electro luminescence (EL). The display device 5 displays, via a video cable 5a, various kinds of information including an image that is subjected to the predetermined image processing by the information processing apparatus 3. Accordingly, an operator is able to observe a desired position inside the subject and determine symptoms by operating the endoscope 2 while viewing the image (in-vivo image) displayed by the display device 5.

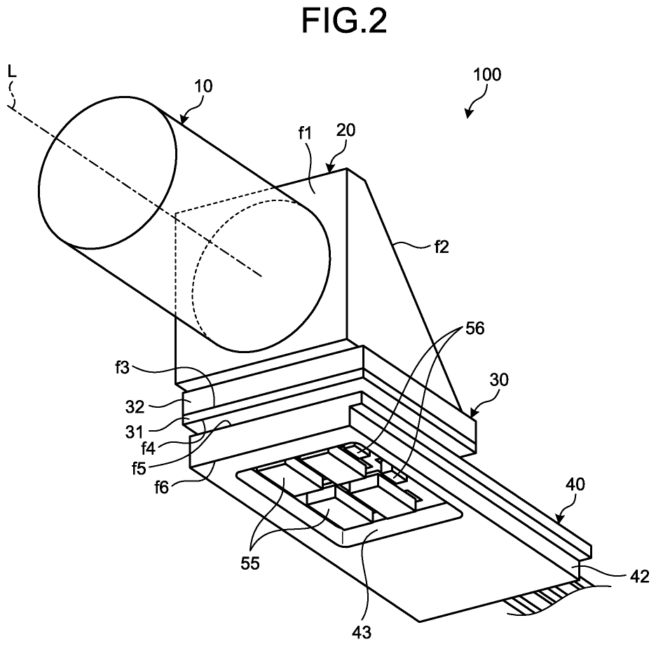

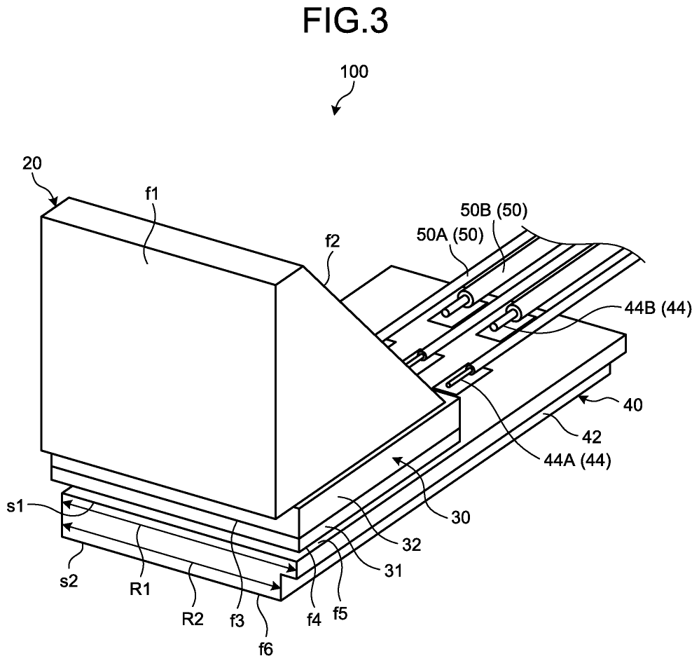

An imaging unit 100 used in the endoscope system 1 will be described in detail below. FIG. 2 is a perspective view of the imaging unit 100 that is arranged in the distal end portion 6a of the endoscope 2 illustrated in FIG. 1. FIG. 3 is a perspective view of the imaging unit 100 viewed from a direction different from FIG. 2. In FIG. 3, illustration of an objective optical system 10 is omitted. In the present specification, a side at the distal end portion 6a of the endoscope 2 will be referred to as a distal end side, and a side at which cables 50 extend will be referred to as a proximal end side.

The imaging unit 100 includes the objective optical system 10 that includes a plurality of objective lenses, a prism 20 that reflects light condensed by the objective optical system 10, a semiconductor package 30 that includes an image sensor 31 configured to generate an electrical signal by receiving light incident from the prism 20 and performing photoelectric conversion on the received light and that includes connection electrodes 33 formed on a back surface f4 thereof, a multi-layer substrate 40 that includes, on a top surface f5 side, connection terminals 41 connected to the connection electrodes 33 via conductive members, such as bumps 34, and the cables 50 that are connected to cable connection electrodes 44 formed on the top surface f5 side of the multi-layer substrate 40 via conductive members, such as soldering (not illustrated).

The semiconductor package 30 is constructed such that a glass 32 is attached to the image sensor 31. Light that enters through a surface f1 of the prism 20 from the objective optical system 10 and that is reflected by a surface f2 enters, via the glass 32, a top surface f3 (light receiving surface) of the image sensor 31 that includes a light-receiving unit. The connection electrodes 33 and the bumps 34 formed of soldering or the like are formed on the back surface f4 of the light receiving surface of the image sensor 31. It is preferable that the semiconductor package 30 is a chip size package (CSP) that is constructed by arranging wires, forming electrodes, performing resin sealing, and performing dicing on an imaging element chip in a wafer state such that a size of the imaging element chip eventually becomes equal to the size of the semiconductor package 30. Further, the semiconductor package 30 is what is called a transverse type, in which the surface f3 that is the light receiving surface of the image sensor 31 is placed parallel to an optical axis L of the objective optical system 10.

The connection terminals 41 connected to the connection electrodes 33 of the image sensor 31 are formed on a distal end side of the top surface f5 of the multi-layer substrate 40, and the cable connection electrodes 44 connected to the cables 50 are formed on a proximal end side. The cable connection electrodes 44 include a cable connection electrode 44A for connecting a cable 50A and a cable connection electrode 44B for connecting a cable 50B, and the cable connection electrode 44A and the cable connection electrode 44B are arranged in a hound's tooth manner, for example.

A concave portion 43, in which electronic components 55 and electronic components 56 are mounted, is formed in a region on a back surface f6 side of the multi-layer substrate 40, the region corresponding to a region where the connection terminals 41 are formed. Further, a stepped portion 42 is formed from a distal end side to a proximal end side on the back surface f6 side of the multi-layer substrate 40 such that a width R1 of a side S1 that is located on the top surface f5 of the multi-layer substrate 40 and that is perpendicular to the optical axis L of the objective optical system 10 (see FIG. 3) becomes longer than a width R2 of a side S2 that faces the side S1 and that is located on the back surface f6 of the multi-layer substrate 40.

As the multi-layer substrate 40, a ceramic substrate, a glass epoxy substrate, a glass substrate, a silicon substrate, or the like is used. From the viewpoint of improvement in reliability of connection to the semiconductor package 30, it is preferable to adopt a substrate, such as a silicon substrate or a ceramic substrate, which is made of a material having a thermal expansion rate similar to that of the material of the semiconductor package 30.

It is preferable that a height h1 of the concave portion 43 formed in the back surface f6 of the multi-layer substrate 40 is equal to a height h2 of the stepped portion 42. For example, if a ceramic substrate is used as the multi-layer substrate 40, a plurality of green sheets are laminated and integrated by applying heat and pressure and thereafter fired. In this case, if the height of the concave portion 43 and the height of the stepped portion 42 are equal to each other, it is sufficient to use only two kinds of green sheets in different shapes, so that a manufacturing process can be reduced.

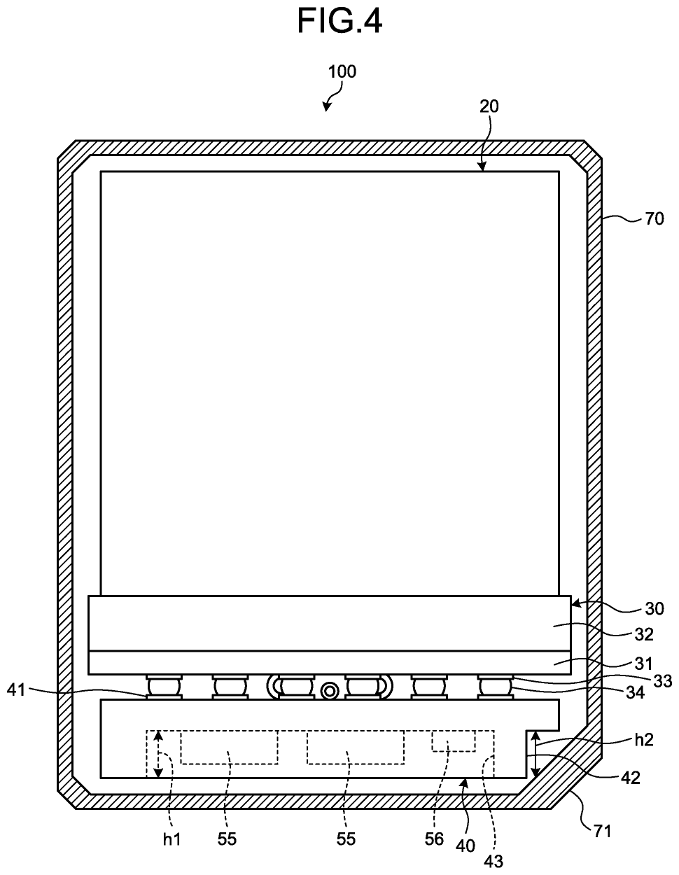

FIG. 4 is a cross-sectional view of the imaging unit 100 illustrated in FIG. 2 in a case where the imaging unit 100 is housed in a frame member 70 (a cross-section of the surface f1 of the prism 20). FIG. 5 is a diagram illustrating arrangement of built-in components on a distal end side of the bending portion 6b of the endoscope 2 illustrated in FIG. 1.

The frame member 70 that has an inner wall and an outer wall formed in shapes conforming to an outer shape of the imaging unit 100, i.e., outer shapes of the prism 20, the semiconductor package 30, and the multi-layer substrate 40, and that holds the imaging unit 100 inside thereof. Therefore, a notch 71 is formed in the frame member 70 in a region adjacent to the stepped portion 42 formed in the multi-layer substrate 40.

As illustrated in FIG. 5, in the insertion portion of the endoscope 2, the treatment tool channel 90 and the imaging unit 100 are arranged in a central portion and the illumination channels 91 are arranged in upper and lower portions. The imaging unit 100 is arranged such that the notch 71 of the frame member 70 is located adjacent to a cladding tube 80.

The imaging unit 100 according to the first embodiment is configured such that the stepped portion 42 is formed from the distal end side to the proximal end side on the back surface f6 side of the multi-layer substrate 40 and the frame member 70 holding the imaging unit 100 is arranged such that an outer peripheral portion adjacent to the stepped portion 42, i.e., the notch 71, is located adjacent to the cladding tube 80, so that it is possible to reduce a diameter of the insertion portion of the endoscope 2.

In the first embodiment as described above, the stepped portion 42 is formed in the multi-layer substrate 40, but embodiments are not limited thereto, and it may be possible to form a tapered notch or the like from the distal end side to the proximal end side on the back surface f6 side of the multi-layer substrate 40 as long as the width R1 of the side S1 that is located on the top surface f5 and that is perpendicular to the optical axis L of the objective optical system 10 becomes longer than the width R2 of the side S2 that is located on the back surface f6 and that faces the side S1.

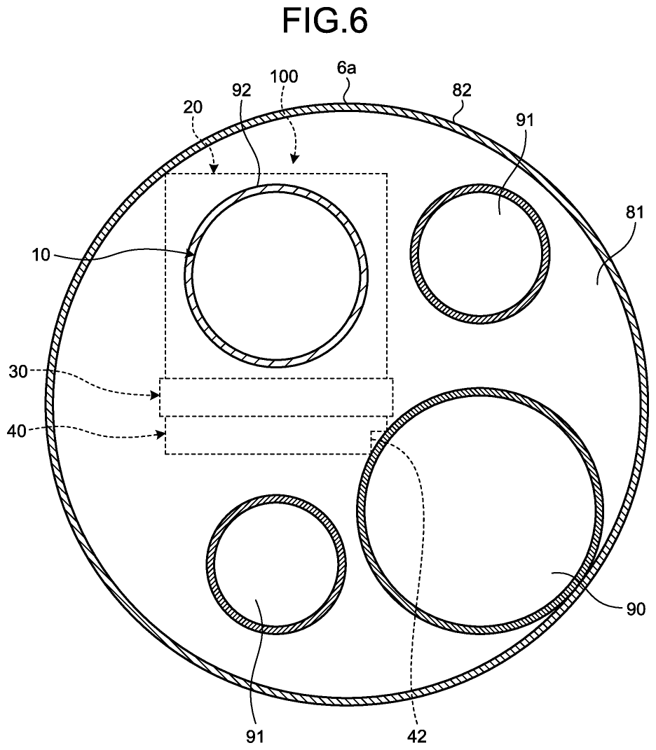

Furthermore, in the first embodiment, the imaging unit 100 is arranged in the insertion portion of the endoscope 2 such that the notch 71 of the frame member 70 is located adjacent to the cladding tube 80, but it may be possible to arrange the imaging unit 100 such that the notch 71 of the frame member 70 is located adjacent to a built-in component. FIG. 6 is a front elevation view of the distal end of the endoscope 2 according to a first modification of the first embodiment of the present disclosure.

In the first modification of the first embodiment, the imaging unit 100 is arranged such that the objective optical system 10 is fitted and fixed to a fitting hole 92 of a distal-end fixing portion 81 that is fitted to a distal-end main body 82 from a front side. The imaging unit 100 is arranged such that the stepped portion 42 is located adjacent to the treatment tool channel 90.

In the first modification of the first embodiment, the stepped portion 42 of the imaging unit 100 is arranged so as to be located adjacent to the built-in component housed in the insertion portion, so that it is possible to reduce the diameter of the insertion portion of the endoscope 2.

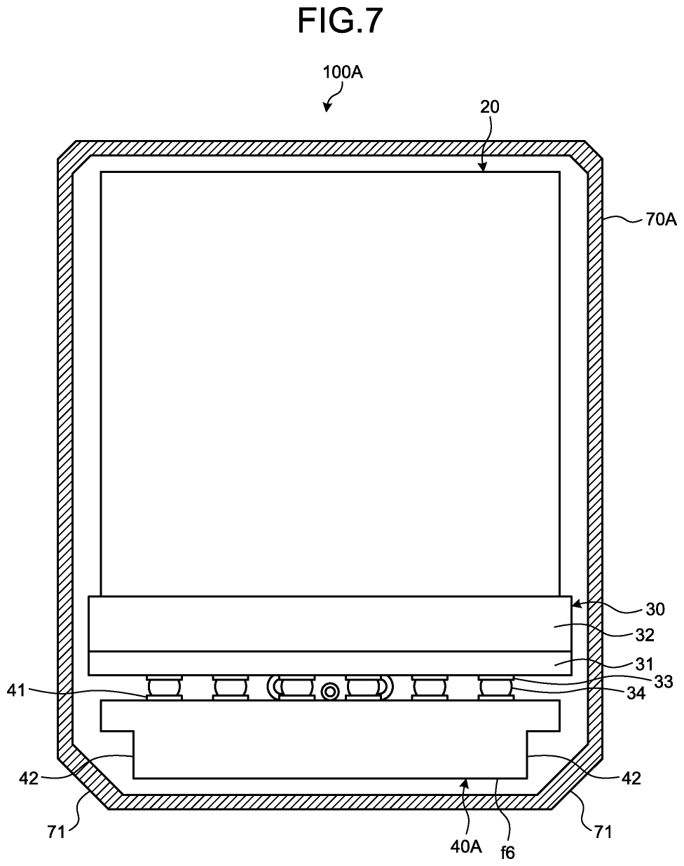

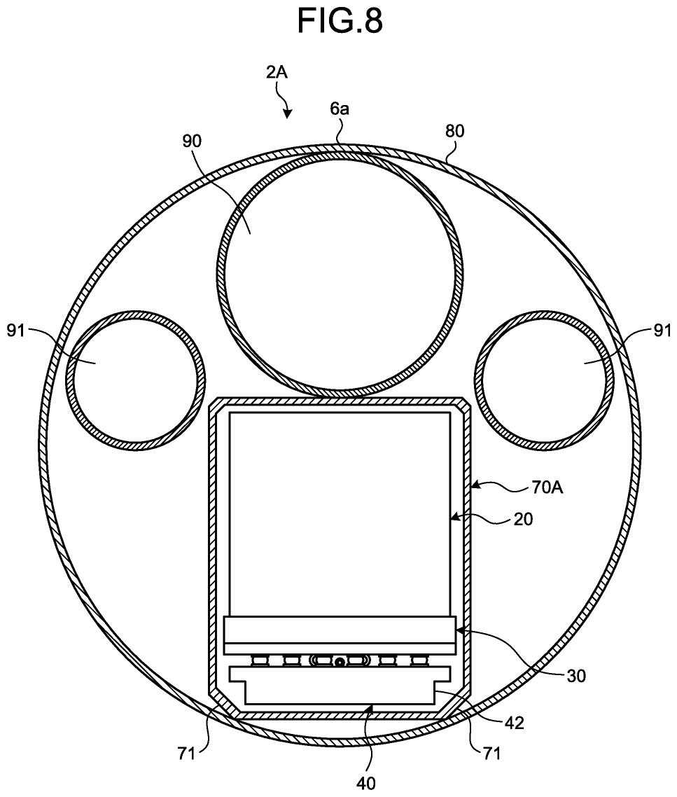

Furthermore, while in the imaging unit 100 of the first embodiment, the stepped portion 42 is formed from the distal end side to the proximal end side on one side of the back surface f6 of the multi-layer substrate 40, it may be possible to form the stepped portion 42 on each of two sides facing one another. FIG. 7 is a front view of an imaging unit 100A according to a second modification of the first embodiment of the present disclosure in a case where the imaging unit 100A is housed in a frame member 70A. FIG. 8 is a cross-sectional view of a distal end of the bending portion 6b of an endoscope 2A according to the second modification of the first embodiment of the present disclosure.

In the imaging unit 100A, the stepped portion 42 is formed from a distal end side to a proximal end side on each of two sides that are located on the back surface f6 of a multi-layer substrate 40A and that are parallel to the optical axis L of the objective optical system 10. Further, the notches 71 are formed in the frame member 70A in respective regions adjacent to the stepped portions 42 of the multi-layer substrate 40A.

As illustrated in FIG. 8, in the insertion portion of the endoscope 2A, the treatment tool channel 90 and the imaging unit 100A are arranged in a central portion and the illumination channels 91 are arranged in left and right portions. The imaging unit 100A is arranged such that the two notches 71 of the frame member 70A are located adjacent to the cladding tube 80.

In the second modification of the first embodiment, the two notches 71 of the frame member 70A holding the imaging unit 100A are arranged so as to be located adjacent to the cladding tube 80, so that it is possible to reduce a diameter of the insertion portion of the endoscope 2A.

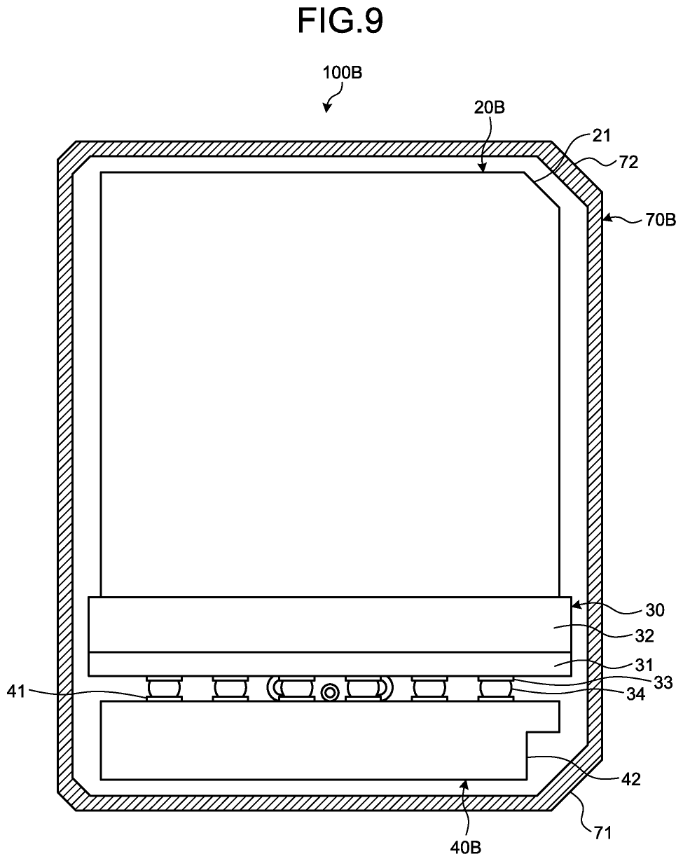

In addition, it is possible to form a notch on a top surface side of the prism 20. FIG. 9 is a front view of an imaging unit 100B according to a third modification of the first embodiment of the present disclosure in a case where the imaging unit 100B is housed in a frame member 70B.

In the imaging unit 100B, the stepped portion 42 is formed from a distal end side to a proximal end side on one side that is located on the back surface f6 of a multi-layer substrate 40B and that is parallel to the optical axis L of the objective optical system 10, and a notch 21 is formed from a distal end side to a proximal end side on one side of a top surface of a prism 20B at a side where the stepped portion 42 of the multi-layer substrate 40B is formed. Further, the notch 71 and a notch 72 are formed, in the frame member 70B holding the imaging unit 100B, in respective regions adjacent to the stepped portion 42 of the multi-layer substrate 40B and the notch 21 of the prism 20B.

In the third modification of the first embodiment, the notch 71 and the notch 72 of the frame member 70B holding the imaging unit 100B are arranged so as to be located adjacent to the cladding tube 80 or a built-in component of the insertion portion, so that it is possible to reduce the diameter of the insertion portion of the endoscope.

### Second Embodiment

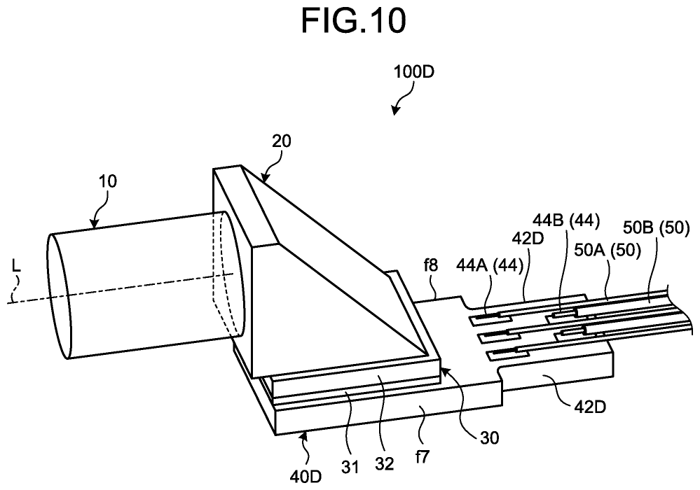

FIG. 10 is a perspective view of an imaging unit 100D according to a second embodiment of the present disclosure. FIG. 11 is a perspective view the imaging unit 100D viewed from a direction different from FIG. 10.

In the imaging unit 100D, notches 42D are formed on a proximal end side of a multi-layer substrate 40D, for example, are formed in a side surface f7 and a side surface f8 of a multi-layer substrate 40D including a region where the cable connection electrodes 44 for connecting the cables 50 are formed, the side surface f7 and the side surface f8 being parallel to the optical axis L of the objective optical system 10. As illustrated in FIG. 11, the notches 42D are formed such that a width R3 of a side surface f9 that is located on a distal end side of the multi-layer substrate 40D and that is perpendicular to the optical axis L of the objective optical system 10 becomes longer than a width R4 of a side surface f10 on a proximal end side.

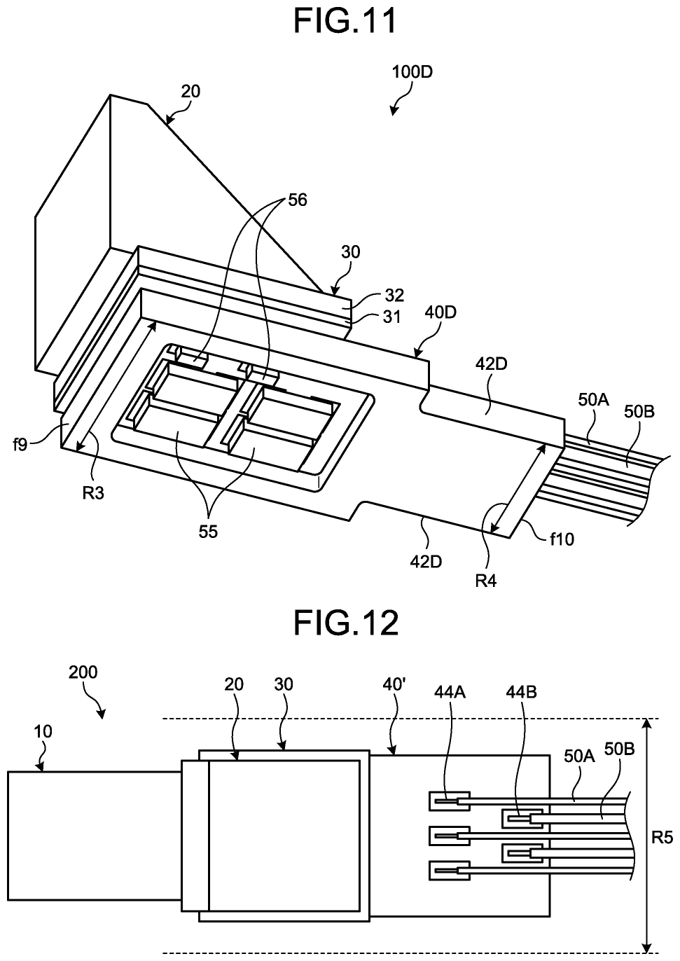

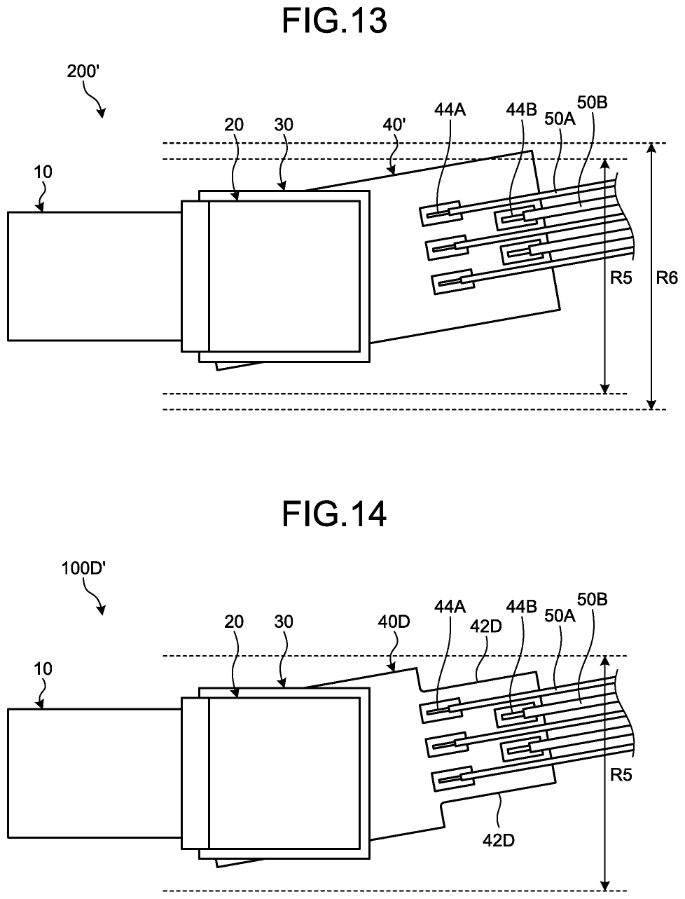

FIG. 12 is a diagram for explaining a conventional imaging unit 200 (mounting misalignment has not occurred). FIG. 13 is a diagram for explaining a conventional imaging unit 200′ (mounting misalignment has occurred). FIG. 14 is a diagram for explaining an imaging unit 100D′ according to the second embodiment of the present disclosure (mounting misalignment has occurred). The imaging unit 200 and the imaging unit 200′ illustrated in FIG. 12 and FIG. 13 are different from the imaging unit 100D according to the second embodiment of the present disclosure only in that notches 42D are not formed in multi-layer substrates 40′.

The imaging unit 200 illustrated in FIG. 12 in which mounting positions of the multi-layer substrate 40′ and the semiconductor package 30 are not misaligned does not interfere with an inner wall of the insertion portion (cladding tube) that has a small diameter (inner diameter R5) as indicated by a dotted line when the objective optical system 10 or the frame member is arranged inside the insertion portion of the endoscope.

However, as illustrated in FIG. 13, in some cases, the mounting positions of the multi-layer substrate 40′ and the semiconductor package 30 may be misaligned in a manufacturing process. In this case, interference with the inner wall of the insertion portion (cladding tube) that has the small diameter (inner diameter R5) occurs; therefore, the insertion portion (cladding tube) has been designed so as to have a larger diameter (R6) to improve yields.

In the second embodiment, the notches 42D are formed on the proximal end side of the multi-layer substrate 40D, so that, as illustrated in FIG. 14, even when mounting positions of the multi-layer substrate 40D and the semiconductor package 30 are misaligned, interference with the insertion portion (cladding tube) that has the small diameter (inner diameter R5) does not occur. Therefore, it is possible to reduce the diameter of the insertion portion.

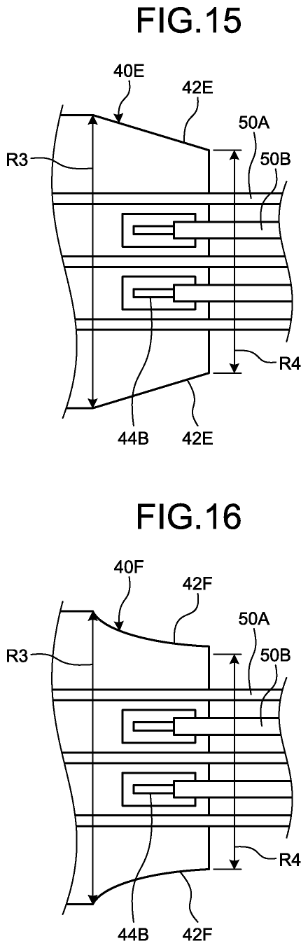

While the notches 42D are formed in stepped shapes in the second embodiment, embodiments are not limited thereto. FIG. 15 is a partial top view of a multi-layer substrate 40E according to a first modification of the second embodiment of the present disclosure. FIG. 16 is a partial top view of a multi-layer substrate 40F according to a second modification of the second embodiment of the present disclosure.

The notches formed on a proximal end side of the multi-layer substrate may be notches 42E in tapered shapes as illustrated in FIG. 15 or notches 42F in circular arc shapes as illustrated in FIG. 16 as long as the width R3 of the side surface f9 of each of the multi-layer substrates 40E and 40F is longer than the width R4 of the side surface f10 on the proximal end side.

### Third Embodiment

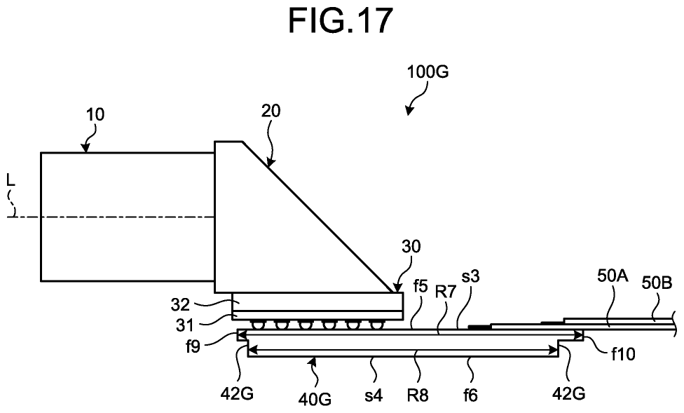

FIG. 17 is a side view of an imaging unit 100G according to a third embodiment of the present disclosure.

In the imaging unit 100G, notches 42G are formed in the back surface f6 of a multi-layer substrate 40G such that a width R7 of a side S3 that is located on the top surface f5 and that is parallel to the optical axis L of the objective optical system 10 becomes longer than a width R8 of a side S4 that is located on the back surface f6 and that faces the side S3. The notches 42G are formed in the entire side surface f9 and the entire side surface surface f10 that are perpendicular to the optical axis L of the objective optical system 10.

In some cases, mounting positions of the multi-layer substrate 40G and the semiconductor package 30 may be misaligned in a Z-direction (height direction) in a manufacturing process of the imaging unit 100G. In the third embodiment, the notches 42G are formed in the entire side surface f9 and the entire side surface f10, which are perpendicular to the optical axis L of the objective optical system 10, in the multi-layer substrate 40G; therefore, interference with an inner wall of the insertion portion (cladding tube) that has a small diameter does not occur, so that it is possible to reduce the diameter of the insertion portion.

In the third embodiment, the notches 42G are formed in the side surface f9 on the distal end side of the multi-layer substrate 40G and in the side surface f10 on the proximal end side; however, from the view point of reducing the diameter of the insertion portion, it is sufficient to form the notch 42G at least in the side surface f10 on the proximal end side.

Furthermore, while the notches 42G in stepped shapes are formed in the third embodiment, embodiments are not limited thereto. It may be possible to form notches in tapered shapes as long as the width R7 of the side S3 of the multi-layer substrate 40G becomes longer than the width R8 of the side S4 on the back surface f6.

The imaging unit according to the present disclosure is useful for an endoscope system whose diameter needs to be reduced.

According to one aspect of the present disclosure, it is possible to reduce diameters of an imaging unit and an endoscope with simple structure.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An imaging unit comprising:
an optical system including a plurality of lenses;
a prism configured to reflect light condensed by the optical system;
a semiconductor package including an image sensor configured to generate an electrical signal by receiving light incident from the prism and performing photoelectric conversion on the received light, and including a connection electrode on a back surface of the semiconductor package; and
a multi-layer substrate including a connection terminal on a top surface of the multi-layer substrate, the connection electrode being connected to the connection electrode via a conductive member, wherein
a concave portion in which an electronic component is mounted is formed in a region on a back surface of the multi-layer substrate, the region corresponding to a region where the connection terminal is formed,
a stepped portion is formed from a distal end side to a proximal end side on a back surface side of the multi-layer substrate so as to make a width of a first side that is located on the top surface of the multi-layer substrate and that is perpendicular to an optical axis of the optical system to become longer than a width of a second side that is located on the back surface of the multi-layer substrate and that faces the first side, and
a height of the concave portion and a height of the stepped portion are equal to each other.

2. The imaging unit according to claim 1, wherein a notch is formed on a top surface side of the prism.

3. An endoscope comprising:
the imaging unit according to claim 1; and
an insertion portion including a distal end portion in which a tubular main body made of a rigid material is covered by a cladding tube, the insertion portion being configured to be inserted into a subject, wherein
the imaging unit is located in an internal space of the cladding tube of the insertion portion.

4. The endoscope according to claim 3, wherein the stepped portion is located adjacent to one of the cladding tube and a built-in component housed in the insertion portion.

5. An endoscope comprising:
the imaging unit according to claim 1;
a frame member that has an inner wall and an outer wall formed in shapes conforming to an outer shape of the imaging unit, the frame member being configured to hold the imaging unit; and
an insertion portion including a distal end portion in which a tubular main body made of a rigid material is covered by a cladding tube, the insertion portion being configured to be inserted into a subject, wherein
the imaging unit held by the frame is located in an internal space of the cladding tube of the insertion portion.

6. The endoscope according to claim 5, wherein an outer peripheral portion of the frame member, which is located adjacent to the stepped portion, is located adjacent to one of the cladding tube and a built-in component housed in the insertion portion.

7. An imaging unit comprising:
an optical system including a plurality of lenses;
a prism configured to reflect light condensed by the optical system;
a semiconductor package including an image sensor configured to generate an electrical signal by receiving light incident from the prism and performing photoelectric conversion on the received light, and including a connection electrode on a back surface of the semiconductor package;
a multi-layer substrate including a connection terminal on a top surface of the multi-layer substrate, the connection electrode being connected to the connection electrode via a conductive member, wherein
a notch is formed on a proximal end side of the multi-layer substrate so as to make a width of a first side surface that is located on a distal end side of the multi-layer substrate and that is perpendicular to an optical axis of the optical system to become longer than a width of a second side surface on the proximal end side of the multi-layer substrate.

8. The imaging unit according to claim 7, wherein
a cable connection electrode for connecting a cable is formed in the multi-layer substrate and on a proximal end side of a region where the connection terminal is formed, and
the notch is formed in a side surface of the multi-layer substrate, the side surface including the region where the cable connection electrode is formed, the side surface being parallel to the optical axis of the optical system.

9. An imaging unit comprising:
an optical system including a plurality of lenses;
a prism configured to reflect light condensed by the optical system;
a semiconductor package including an image sensor configured to generate an electrical signal by receiving light incident from the prism and performing photoelectric conversion on the received light, and including a connection electrode on a back surface of the semiconductor package; and
a multi-layer substrate including a connection terminal on a top surface of the semiconductor package, the connection electrode being connected to the connection electrode via a conductive member, wherein
a notch is formed on a proximal end side on a back surface side of the multi-layer substrate so as to make a length of a first side that is located on the top surface side of the multi-layer substrate and that is parallel to an optical axis of the optical system to become longer than a length of a second side that is located on the back surface side of the multi-layer substrate and that faces the first side.
